(19) United States
(12) Patent Application Publication
(10) Pub. No.: US 2024/0358628 A1
(43) Pub. Date: Oct. 31, 2024

(54) CYANOBACTERIAL POLYSACCHARIDE COMPOSITIONS

(71) Applicant: HELIOBIOSYS, INC, Woodside, CA (US)

(72) Inventors: Rocco Mancinelli, Woodside, CA (US); David Smernoff, Portola Valley, CA (US)

(21) Appl. No.: 18/688,032

(22) PCT Filed: Dec. 21, 2022
(86) PCT No.: PCT/US2022/082109

§ 371 Date: Feb. 29, 2024

Related U.S. Application Data
(60) Provisional application No. 63/265,984, filed on Dec. 23, 2021.

(51) Int. Cl. A61K 8/99; A61K 8/73; A61Q 19/08

(52) U.S. Cl. CPC A61K 8/99; A61K 8/73; A61Q 19/08; A61K 2800/412

(57) ABSTRACT

Provided herein are cyanobacterial polysaccharide compositions. Also provided are methods of using polysaccharides for applications such as topical personal care products, cosmetics, and wrinkle reduction compositions. The disclosure also provides novel cyanobacterial compositions useful for improving the health and appearance of skin.

### CLAIM FOR PRIORITY

This application claims the benefit of priority of U.S. Application Ser. No. 63/265,984, filed Dec. 23, 2021, which is hereby incorporated by reference in its entirety.

### FIELD OF THE DISCLOSURE

The disclosure resides in the fields of health and beauty, cosmetic ingredients and aquaculture.

### BACKGROUND OF THE DISCLOSURE

Polysaccharides are long chain polymeric carbohydrates composed of monosaccharide units bound together by glycosidic linkages. The carbohydrate can react with water (hydrolysis) with enzymes as catalysts to produce constituent sugars (monosaccharides, or oligosaccharides). Polysaccharide structures include linear as well as branched and highly branched structure. Examples include cellular storage polysaccharides such as starch, glycogen and galactogen and structural polysaccharides such as cellulose and chitin.

Polysaccharides are often quite heterogeneous, containing slight modifications of repeating units. Depending on the structure, polysaccharides can have distinct properties from their monosaccharide building blocks. They may be amorphous or even insoluble in water. When all the monosaccharides in a polysaccharide are the same type, the polysaccharide is called a homopolysaccharide or homoglycan, but when more than one type of monosaccharide is present they are called heteropolysaccharides or heteroglycans.

Natural polysaccharides are generally composed of simple carbohydrates (saccharides) called monosaccharides with the general formula (CH₂O)n where n is three or more. Examples of monosaccharides include glucose, fructose, and mannose. Polysaccharides, meanwhile, have a general formula of Cx(H₂O)y where x is usually a large number between 200 and 2500. When the repeating units in the polymer backbone are six-carbon monosaccharides, as is often the case, the general formula simplifies to (C₆H₁₀O₅)n, where typically 40≤n≤3000.

Two monosaccharides can be linked together to form a “double” sugar or disaccharide. Common disaccharides include sucrose, which is common table sugar (glucose+fructose); lactose, which is the major sugar in milk (glucose+galactose); and maltose, which is the product of starch digestion (glucose+glucose). Although the process of linking two monomers is complex, the end result in each case is the loss of a hydrogen atom (H) from one of the monosaccharides and a hydroxyl group (OH) from the other. The resulting linkage between the sugars is called a glycosidic bond. The molecular formula of each of these disaccharides is C₁₂H₂₂O₁₁ is the same as 2 C₆H₁₂O₆—H₂O. All sugars are very soluble in water because of their many hydroxyl groups. Although not as concentrated a fuel as fats, sugars are the most important source of energy for life.

### SUMMARY

The present disclosure relates to polysaccharides and biomass produced from cyanobacteria grown under phototrophic conditions. Unique to the present disclosure is that the polysaccharides are produced by a patented consortium of marine cyanobacteria (Mancinelli, R. L., and D. T. Smernoff. 2015. Compositions and methods for culturing microorganisms. Patent U.S. Pat. No. 9,034,632 B2.) that produces unique polysaccharides. Representative polysaccharides include those present in the cells of cyanobacteria as well as excreted polysaccharides, or exopolysaccharides. In addition to the polysaccharides themselves, such as in an isolated, purified, or semi-purified form, the disclosure includes a variety of compositions containing one or more cyanobacterial polysaccharides as disclosed herein. The compositions include nutraceutical, cosmeceutical, industrial and pharmaceutical compositions which may be used for a variety of indications and uses as described herein.

Because the EPS is produced by a consortium, the members of which all make EPS, the result is a mixture of extracellular polysaccharides from all of the three members. It is known that the exact structure and composition of an EPS produced by an organism is influenced by its environment. It is also known that when the consortium is growing together, they grow differently than they do in monoculture (e.g., grow faster, to a higher biomass and are resistant to perturbation, etc.). These differences in the growth conditions (environment) results in individual EPSs that are probably different than what they produce in monoculture. It is also known that EPSs interact and react with each other when mixed together as occurs in the consortium (Sekar, N. et al., 2021, Chapter 14—Cyanobacterial extracellular polymeric substances (EPS): Biosynthesis and their potential applications. In. Microbial and Natural Macromolecules: Synthesis and Applications, Pages 349-369, Surajit Das and Hirak Ranjan Dash, editors, published by ScienceDirect® a registered trademark of Elsevier B. V.). This results in a large extracellular polysaccharide that is comprised of the individual extracellular polysaccharides now essentially entangled and held together by ionic interactions.

Other compositions include those containing one or more cyanobacterial polysaccharides and a suitable carrier or excipient for topical or oral administration.

The present disclosure also relates to cyanobacteria for formulation in skin care products as a composition of the disclosure. The disclosure thus provides highly desirable compositions of cyanobacterial cells that provide delivery or high value cosmeceutical ingredients such as carotenoids, phycocyanins, polyunsaturated fatty acids, moisturizing polysaccharides, superoxide dismutase, mycosporine like amino acids and other components.

The disclosure provides the insight that various species of cyanobacteria, when grown under phototrophic conditions, allow for the production of biomass high in cosmeceutical/nutraceutical value as products and ingredients of a skin care formulation. In addition, anti-inflammatory, moisturizing polysaccharides are produced at varying concentrations during culture and can be purified from the culture medium.

The disclosure further relates to methods of producing or preparing cyanobacterial polysaccharides. In some cases the methods involve incorporation of exogenous sugars into polysaccharides to produce polysaccharides distinct from those typically made by the cyanobacteria.

The disclosure further relates to methods of growing, producing and preparing cyanobacterial biomass. In some disclosed methods, the cyanobacteria can rapidly produce the polysaccharides when grown under phototrophic conditions.

In another aspect, the disclosure relates to compositions for topical application, such as a composition for application to human skin comprising a polysaccharide isolated from cells of the genera Synechococcus, and or Cyanothece. In some examples, the composition comprises an exopolysaccharide that is excreted outside the cyanobacterial cell. In other examples, the composition comprises a polysaccharide that is part of a cyanobacterial cell, or a homogenate thereof. In other examples, the polysaccharide is contained within cyanobacterial cells, or a homogenate thereof a cyanobacterial cell, or a homogenate thereof.

In additional examples, the composition is that of a cosmetic or other skin care product. Such products may contain one or more cyanobacterial polysaccharides, or a cyanobacterial cell homogenate, a topical carrier, and/or a preservative. In some examples, the carrier may be any carrier suitable for topical application, such as, but not limited to, use on human skin or human mucosal tissue. In other examples, the composition may contain a purified cyanobacterial polysaccharide, such as an exopolysaccharide, and a topical carrier. Exemplary carriers include liposome formulation, biodegradable microcapsule, lotion, spray, aerosol, dusting powder, biodegradable polymer, mineral oil, liquid petroleum, white petroleum, propylene glycol, polyoxyethylene, polyoxypropylene compound, emulsifying wax, sorbitan monostearate, polysorbate 60, cetyl ester wax, cetearyl alcohol, 2-octyldodecanol, benzyl alcohol, cyclomethicone, cyclopentasiloxane and water. Exemplary preservatives include, but are not limited to, diiodomethyl-p-tolylsulfone, 2-Bromo-2-nitropropane-1,3-diol, cis isomer 1-(3-chloroallyl)-3,5,7-triaza-1-azoniaadamantane chloride, glutaraldehyde, 4,4-dimethyl oxazolidine, 7-Ethylbicyclooxazolidine, methyl paraben, sorbic acid, Germaben II, and disodium EDTA.

As a cosmeceutical, the composition may contain a cyanobacterial polysaccharide or homogenate and other component materials typically found in cosmetics. In some examples, the component material may be that of a fragrance, a colorant (e.g., black or red iron oxide, etc.), a sunblock (e.g., titanium dioxide, zinc oxide, etc.), and a mineral or metallic additive.

In other aspects, the disclosure includes methods of preparing or producing a cyanobacterial polysaccharide. In some aspects relating to an exopolysaccharide, the disclosure includes methods that separate the exopolysaccharide from other molecules present in the medium used to culture exopolysaccharide producing cyanobacteria. In some examples, separation includes removal of the cyanobacterial cells from the culture medium containing the exopolysaccharide, after the cyanobacterial culture has been cultured for a period of time. The methods may be practiced with cyanobacterial polysaccharides other than exopolysaccharides.

In one example, the disclosure includes a method of producing an exopolysaccharide, wherein the method comprises culturing cyanobacteria in a photobioreactor or outdoor cultivation system, separating the cyanobacterial cells from culture medium, wherein the culture medium contains the exopolysaccharide, and separating the exopolysaccharide from other molecules present in the culture medium. In other examples, the exopolysaccharide is shed or secreted from the cyanobacterial cells into the surrounding culture media.

The cyanobacteria of the disclosure may be that of any species of cyanobacteria that is capable of producing exopolysaccharide (high molecular weight polysaccharide that is secreted into the culture medium) when cultured under photototrophic conditions. In some examples, the cyanobacteria are from the genera Synechococcus or Cyanothece. Some species in these genera have been discovered to secrete large amounts of polysaccharide into their surrounding growth medium. Non-limiting examples of species within a cyanobacterial genus of the disclosure include Synechococcus CCMP 1333, Synechococcus PCC 7002, and Cyanothece Miami BG 043511.

Other examples of the method includes the separation of an exopolysaccharide from other molecules present in the culture medium by tangential flow filtration. Alternatively, the methods may be practiced by separating an exopolysaccharide from other molecules present in the culture medium by alcohol precipitation. Non-limiting examples of alcohols to use include ethanol, isopropanol and methanol.

In other examples, a method may further comprise treating a polysaccharide or exopolysaccharide with a protease to degrade polypeptide (or proteinaceous) material attached to, or found with, the polysaccharide or exopolysaccharide. The methods may optionally comprise separating the polysaccharide or exopolysaccharide from proteins, peptides, and amino acids after protease treatment.

In other examples, a method of formulating a cosmeceutical composition is disclosed. As one non-limiting example, the composition may be prepared by adding separated polysaccharides, or exopolysaccharides, to homogenized cyanobacterial cells before, during, or after homogenization. Both the polysaccharides and the cyanobacterial cells may be from a culture of cyanobacterial cells in suspension and under conditions allowing or permitting cell division. The culture medium containing the polysaccharides is then separated from the cyanobacterial cells followed by (1) separation of the polysaccharides from other molecules in the medium and optionally, (2) homogenization of the cells.

Other compositions of the disclosure may be formulated by subjecting a culture of cyanobacterial cells and soluble exopolysaccharide to tangential flow filtration until the composition is substantially free of cellular and medium components. Alternatively, a polysaccharide is prepared after proteolysis of polypeptides present with the polysaccharide. The polysaccharide and any contaminating polypeptides may be that of a culture medium separated from cyanobacterial cells in a culture thereof.

In a further aspect, the disclosed disclosure includes a composition comprising particulate polysaccharides. The polysaccharides may be from any cyanobacterial source, and with any level of sulfation, as described herein. The composition may be sterile or substantially free of endotoxins and/or proteins in some examples. In other examples, the composition further comprises hyaluronic acid or another agent suitable or desirable for treatment of skin. The particles in some examples are generated by first purifying the polysaccharide away from biomass, then drying the purified polysaccharide into a film, and then homogenizing and/or grinding the film into smaller particles. In alternative examples the polysaccharide can be concentrated using alcohol precipitation before or after the polysaccharide has been separated from cells.

In some examples, the polysaccharides are in the form of a purified material that was dried to be completely or partially insoluble in water. The purified material has been separated from cell biomass, for example as described in Example 2. In such purified form the polysaccharide is at least 50% polysaccharide by weight, or at least above 75% polysaccharide by weight. In some examples, the dried polysaccharide particles are in mixture with a non-aqueous solvent or material. In other examples, the dried polysaccharide particles are partially soluble such that they are from less than about 70% to less than about 1% soluble in water.

In additional examples, the polysaccharide particles increase in volume, or swell, on contact with water or water vapor. Thus, the volume of the polysaccharide particles increases compared to its anhydrous or partially hydrated volume before exposure to the water or water vapor. In some examples, the particles increase in volume by an amount selected from at least about 5% to at least about 5000%.

In some examples, the polysaccharide compositions described herein further comprise at least one ingredient selected from the group consisting of beta carotene, lutein, astaxanthin, vitamin C, vitamin E, vitamin A, coenzyme Q10, a peptide, an acylated peptide, oil soluble α-hydroxy acid, an alkyl lactate, salicylic acid, phycocyanin and mycosporine like amino acids. In some cases, the compositions comprise an aqueous solution or micronized particles containing the polysaccharide and at least one other ingredient. In some cases, the particles are of a substantially uniform size. In some examples, the cyanobacterial polysaccharide and at least one ingredient have been subjected to heating, drying and homogenization to form a solution or particles comprising both cyanobacterial polysaccharides and at least one other ingredient.

In other examples, the polysaccharide compositions described herein further comprise at least one ingredient selected from the group consisting of water, sodium hyaluronate, betaine, trisodium EDTA, glycerin, butylene glycol, amphisol K, shea butter, macadamian oil, isocetyl stearate, olive oil, PEG 150 distearate, grancil VX401, glyceryl monostearate, polyethylene, granpowder USQ, grancil PSQ, diocide and fragrance. In some cases, the compositions comprise an aqueous solution or micronized particles containing the polysaccharide and the at least one other ingredient. In some cases, the polysaccharide composition is formulated for topical application.

The disclosure further includes methods for the preparation or manufacture of the polysaccharides in aqueous solution or dried polysaccharide particles. In some examples, the method comprises formulating particles of polysaccharide material into a non-aqueous material. The particles may be formed from a film of dried polysaccharide material, wherein at least a portion (or some proportion) of the film has been made completely or partially insoluble in water. Optionally, the particles are formed by homogenization of the film into particulate form.

In some cases, the film is formed by heating a suspension of polysaccharide material until all or part of the film is insoluble. The heating may be of an aqueous suspension of the material to remove water from the suspension. The polysaccharide in suspension may be from any cyanobacterial source as described herein. Optionally, the polysaccharide in suspension has been isolated from cyanobacterial biomass. Optionally, the polysaccharide in the suspension has been isolated from supernatant of a culture of cyanobacteria.

The disclosure thus includes a method of preparing or manufacturing a composition for topical application, such as for improving the appearance of skin. The method may comprise 1) concentrating an aqueous solution of polysaccharides that have been separated from the biomass by centrifugation and or tangential flow filtration; 2) drying an aqueous suspension of a polysaccharide isolated from cyanobacteria to a solid film, wherein at least some proportion of the film has been made completely or partially insoluble in water; 3) homogenizing the film into particles; and optionally 4) formulating the particles into a non-aqueous material. In some examples the concentrated aqueous polysaccharide solution may comprise a solution suitable for formulation into a topical application. In some examples, the homogenizing is via a method selected from jet milling, ball milling, Retsch® milling, pin milling and milling in a Quadro® device. Optionally, the formulating of the particles into the non-aqueous phase of an oil-in-water emulsion, such as an emulsion suitable for topical application. The non-aqueous phase may comprise an oil suitable for topical application, such as hexadecanoic acid as a non-limiting example. In other cases, the formulating of the particles is into a carrier suitable for topical administration as described herein. In some examples, the particles may be relatively uniform in size or may range in size, but in many examples, the particles have an average size between about 400 and 0.1 microns.

In some alternative examples, a method to render the polysaccharide material insoluble is selected from chemical cross-linking, chemical dehydration through displacement of bound water by an alcohol, precipitation from solution using an alcohol or a ketone or pH, and coating of particles by microencapsulation.

In an additional aspect, the disclosure includes a method of topically applying a composition comprising polysaccharides in particulate form. In some examples, the application is to skin, such as to mammalian or human skin. Alternatively, the application is to lips or wrinkles on human skin, or by injection into skin or a skin tissue. In many examples, the application is to improve the appearance of skin.

In additional examples, a polysaccharide containing composition (optionally with polysaccharides in particulate form) may be used in a method of cosmetic enhancement. In one example, a method may include injecting a polysaccharide produced by cyanobacteria into mammalian skin. In some examples, the polysaccharide is sterile and free of protein.

In further examples, a method to treat skin, such as mammalian or human skin, is disclosed. In some examples, the method is for the treatment of human facial skin or a tissue thereof. Such methods include a method of stimulating collagen and or elastin synthesis in skin by applying a disclosed composition of the disclosure to the skin. Additional methods include a method to reduce the signs of aging or reduce the appearance of aging in human skin by applying a composition of the disclosure to the skin. Non-limiting examples of a sign of aging or an appearance of aging include wrinkles, such as those on the forehead or around the eyes and/or lips, and age spots, that is, yellowish-brown flat spots that appear as large freckles on the skin caused by overactive pigment cells.

Additional examples include the use of a polysaccharide containing composition in a method of reducing the effects of ultraviolet (UV) light or radiation, such as that present in sunlight, on skin or a skin tissue. One non-limiting example is a method of shielding mammalian skin from UV light. The method may comprise applying a composition of the disclosure to skin or a skin tissue in an effective or sufficient amount to shield, at least in part, the skin from UV radiation. In an alternative example, a composition of the disclosure may be applied in an effective or sufficient amount to treat skin that has been damaged by UV radiation.

In addition to the above, application of a composition of the disclosure to human skin may be used in a method of reducing reactive oxygen species (ROS) in the skin or a skin tissue. This is based in part on the insight that the disclosed polysaccharides in combination with other cyanobacterial cell extracts possess anti-oxidant activity. In some examples, the method is used to prevent or treat a disease or unwanted condition associated with ROS or oxidative stress. Non-limiting examples of such a disease or unwanted condition include reducing inflammation or irritation of the skin. In another example, a composition of the disclosure may reduce inflammation by reducing lymphocyte proliferation that is associated with inflammation. In yet another example, a composition of the disclosure may reduce inflammation by reducing the level of cytokine secretion that is associated with inflammation. In some examples, the polysaccharide composition comprises one or more other agents or compounds with anti-oxidant activity. In further examples, the method may be used to lower the level of ROS, or reduce or decrease the amount of damage caused by ROS in skin or a skin tissue. The amount of the composition may be any that is effective or sufficient to produce a desired improvement or therapeutic benefit.

In other aspects, the present disclosure is directed to a method of reducing fine lines and/or wrinkles on human skin, a method of inducing a feel of tightening human skin, a method of reducing transepidermal water loss in human skin, a method of moisturizing human skin, and/or a method of increasing elasticity of human skin. Each method comprises administration of a composition, as disclosed herein, to human skin in an amount and at a frequency sufficient to impart the desired characteristics. The various methods and/or compositions can be combined to provide methods or compositions suitable for imparting multiple characteristics simultaneously.

The details of additional examples of the disclosure are set forth in the following description.

### BRIEF DESCRIPTION OF THE FIGURES

The drawings illustrate generally, by way of example, but not by way of limitation, various embodiments discussed in the present document.

FIG. 1 is a total ion chromatogram (TIC) of the TMS-glycoside derivatives of Purified exopolysaccharide (EPS).

FIG. 2 is a total ion chromatogram (TIC) of the PMAAs derivatives.

FIG. 3A is a size exclusion chromatogram of a sample.

FIG. 3B is a size exclusion chromatograph of six dextran standards.

FIG. 4 is a graph showing results of a colorimetric assay of cellular metabolic activity.

FIG. 5 is a graph showing results of a type I collagen C—Peptide assay.

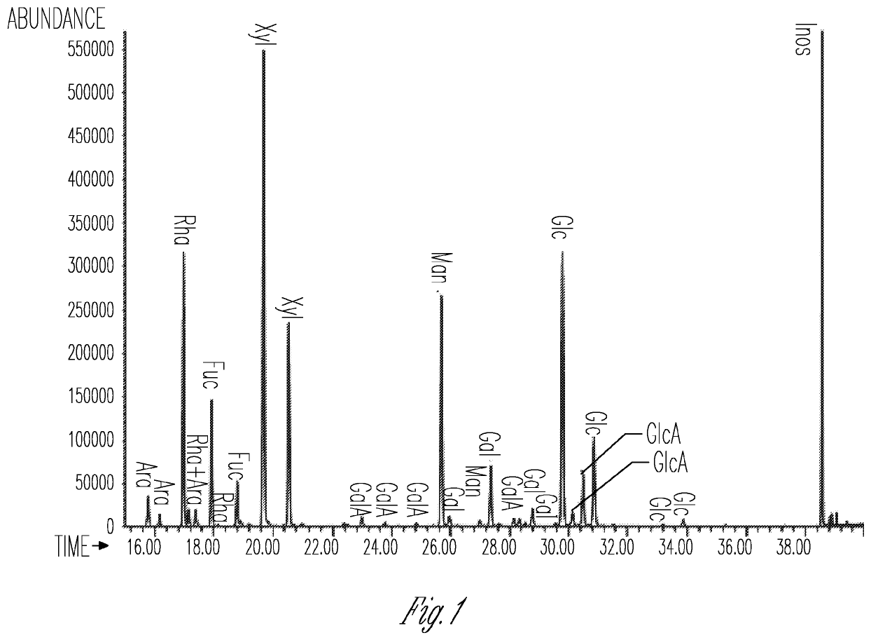

### DETAILED DESCRIPTION

### I. Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “weight-average molecular weight” as used herein refers to Mw, which is equal to ΣMᵢ²nⱼ/ΣMᵢnᵢ, where nᵢ is the number of molecules of molecular weight Mi. In various examples, the weight-average molecular weight can be determined using light scattering, small angle neutron scattering, X-ray scattering, and sedimentation velocity.

“Associates with” means, within the context of a polysaccharide binding fusion protein, one molecule binding to another molecule. Affinity and selectivity of binding can vary when a polysaccharide and a polysaccharide binding protein are in association with each other.

“Axenic” means a culture of an organism that is free from contamination by other living organisms.

The term “biomass” refers to material produced by growth and/or propagation of cells. Biomass may contain cells and/or intracellular contents as well as extracellular material. Extracellular material includes, but is not limited to, compounds secreted by a cell.

“Bioreactor” means an enclosure or partial enclosure in which cells are cultured in suspension.

“Carrier suitable for topical administration” means a compound that may be administered, together with one or more compounds of the present disclosure, and which does not destroy the activity thereof and is nontoxic when administered in concentrations and amounts sufficient to deliver the compound to the skin or a mucosal tissue.

The term “co-culture”, variants thereof such as “co-cultivate”, and “consortium” refer to the presence of two or more types of cells in the same bioreactor or cultivation system. The two or more types of cells may both be microorganisms, such as cyanobacteria, or may be cyanobacteria cultured with different cell types. The culture conditions may be those that foster growth and/or propagation of the two or more cell types or those that facilitate growth and/or proliferation of one, or a subset, of the two or more cells while maintaining cellular growth for the remainder.

“Combination Product” means a product that comprises at least two distinct compositions intended for human administration through distinct routes, such as a topical route and an oral route. In some examples the same active agent is contained in both the topical and oral components of the combination product.

“Conditions favorable to cell division” means conditions in which cells divide at least once every 72 hours.

The term “cultivated”, and variants thereof, refer to the intentional fostering of growth (increases in cell size, cellular contents, and/or cellular activity) and/or propagation (increases in cell numbers) of one or more cells by use of intended culture conditions. The combination of both growth and propagation may be termed proliferation. The one or more cells may be those of a microorganism, such as cyanobacteria. Examples of intended conditions include the use of a defined medium (with known characteristics such as pH, ionic strength, and other nutrients), specified temperature, oxygen tension, carbon dioxide levels, and growth in a bioreactor. The term also refers to the growth or propagation of microorganisms in open ponds or other outdoor cultivation systems with direct human intervention.

“Endopolysaccharide” means a polysaccharide that is retained intracellularly.

“EPA” means eicosapentaenoic acid.

“Exogenously provided” describes a molecule provided to the culture media of a cell culture.

“Exopolysaccharide” means a polysaccharide that is secreted from a cell into the extracellular environment. Some exopolysaccharides are secreted by the cell and become soluble in the culture medium, having lost any physical association with the cells, and are referred to as “soluble exopolysaccharides”. Other exopolysaccharides remain associated with the cell wall and are referred to as “cell wall polysaccharides”. Exopolysaccharides are usually a polymer of monosaccharide units and have high molecular weights, usually with average of 2 million Daltons or greater, although fragments can be smaller in size.

“Filtrate” and “permeate” means the portion of a tangential flow filtration sample that has passed through the filter.

“Fixed carbon source” means molecule(s) containing carbon that are present at ambient temperature and pressure in solid or liquid form (e.g., glucose).

“Glycopolymer” means a biologically produced molecule comprising at least two monosaccharides. Examples of glycopolymers include glycosylated proteins, polysaccharides, oligosaccharides, and disaccharides.

“Homogenate” means cell biomass that has been disrupted and the various components of disrupted cells.

As used herein, the term “lysate” refers to a solution containing the contents of lysed cells.

As used herein, the term “lysis” refers to the breakage of the plasma membrane and optionally the cell wall of a biological organism sufficient to release at least some intracellular contents, often by mechanical, viral or osmotic mechanisms that compromise its integrity. As used herein, the term “lysing” refers to disrupting the cellular membrane and optionally the cell wall of a biological organism or cell sufficient to release at least some intracellular contents.

A compound that can be “metabolized by cells” means a compound whose elemental components are incorporated into products endogenously produced by the cells. For example, a compound containing nitrogen that can be metabolized by cells is a compound containing at least one nitrogen atom per molecule that can be incorporated into a nitrogen containing, endogenously produced metabolite such as an amino acid, or protein.

“Cyanobacteria” means a prokaryotic organism that is capable of performing photosynthesis using chlorophyll a and b. Cyanobacteria are photosynthetic bacteria that live in water and soil, and can manufacture their own food, by fixing atmospheric carbon dioxide into sugars. Because they are bacteria, they are quite small, unicellular, though they often grow in long thin filaments. They have the distinction of being the oldest known fossils, more than 3.5 billion years old. They are one of the largest and most important groups of bacteria on earth.

Many Proterozoic oil deposits are attributed to the activity of cyanobacteria. They are also providers of nitrogen fertilizer in the cultivation of rice and beans. The cyanobacteria have also been useful in shaping the course of evolution and ecological change throughout earth's history. The oxygen atmosphere that we depend on was generated by numerous cyanobacteria during the Archaean and Proterozoic Eras.

The other great contribution of the cyanobacteria is the origin of plants. The chloroplast with which plants make food for themselves is actually a remnant of a cyanobacterium living within the plant's cells. Sometime in the late Proterozoic, or in the early Cambrian, cyanobacteria began to take up residence within certain eukaryote cells, making food for the eukaryote host in return for a home. This event is known as endosymbiosis which led to plant cell chloroplasts.

Because they are photosynthetic and aquatic, cyanobacteria are often called “blue-green algae”. This name is convenient for talking about organisms in the water that make their own food, but does not reflect any relationship between the cyanobacteria and eukaryotic organisms called algae. Cyanobacteria are members of the of the domain Bacteria and are not members of the Eukarya nor the Archaea. It is only the chloroplast in eukaryotic algae to which the cyanobacteria are closely related.

The term “cyanobacterial extract” means any component that can be extracted from cyanobacteria. These components can include, but are not limited to; oils, proteins, chlorophyll, phycocyanin, mycosporine like amino acids, lipids, carbohydrates, phospholipids, polysaccharides, macromolecules, minerals, cell wall, trace elements, carotenoids, and sterols.

The terms “microorganism” and “microbe” are used interchangeably herein to refer to microscopic unicellular organisms.

“Naturally produced” describes a compound that is produced by a wild-type organism.

“Peptide” means a polypeptide of 50 or less amino acids. In some contexts, a peptide is connected to a much larger protein as a fusion protein and is referred to as a peptide to denote its independent domain as a part of the fusion protein.

“Polysaccharide material” is a composition that contains more than one species of polysaccharide, including exopolysaccharide and optionally contaminants such as proteins, lipids, and nucleic acids, such as, for example, a cyanobacterial cell homogenate.

“Polysaccharide” means a compound or preparation consisting of complex carbohydrate polymers of more than two monosaccharides linked together covalently by glycosidic linkages in a condensation reaction. A “polysaccharide”, “endopolysaccharide” or “exopolysaccharide” can be a preparation of polymer molecules that have similar or identical repeating units but different molecular weights within the population. A “predominant species” of the polysaccharide is a polymer species that makes up at least 70% of the polysaccharide population.

“Retentate” means the portion of a tangential flow filtration sample that has not passed through the filter.

“Small molecule” means a molecule having a molecular weight of less than 2000 daltons, in some instances less than 1000 daltons, and in still other instances 500 daltons or less. Such molecules include, for example, heterocyclic compounds, carbocyclic compounds, sterols, amino acids, lipids, carotenoids and polyunsaturated fatty acids.

“Substantially free of protein” means compositions that are of high purity and are substantially free of potentially harmful contaminants, including proteins (e.g., at least National Food (NF) grade, generally at least analytical grade, and more typically at least pharmaceutical grade). Compositions are at least 80, at least 90, at least 98%, at least 99 or at least 99.9% w/w pure of undesired contaminants such as proteins are substantially free of protein. To the extent that a given compound must be synthesized prior to use, the resulting product is typically substantially free of any potentially toxic agents, particularly any endotoxins, that may be present during the synthesis or purification process. Compositions are usually made under GMP conditions. Compositions for parenteral administration are usually sterile and substantially isotonic.

### II. General

Polysaccharides form a heterogeneous group of polymers of different length and composition. They are constructed from monosaccharide residues that are linked by glycosidic bonds. Glycosidic linkages may be located between the C1 (or C2) of one sugar residue and the C2, C3, C4, C5 or C6 of the second residue. A branched sugar results if more than two types of linkage are present in single monosaccharide molecule.

Monosaccharides are simple sugars (saccharides) with one or more hydroxyl groups. Based on the number of carbons (e.g., 3, 4, 5, or 6) a monosaccharide is a triose, tetrose, pentose, or hexose. Pentoses and hexoses can cyclize, as the aldehyde or keto group reacts with a hydroxyl on one of the distal carbons. Examples of monosaccharides are galactose, glucose, and rhamnose.

Polysaccharides are molecules comprising a plurality of monosaccharides covalently linked to each other through glycosidic bonds. Polysaccharides consisting of a relatively small number of monosaccharide units, such as 10 or less, are sometimes referred to as oligosaccharides. The end of the polysaccharide with an anomeric carbon (C1) that is not involved in a glycosidic bond is called the reducing end. A polysaccharide may consist of one monosaccharide type, known as a homopolymer, or two or more types of monosaccharides, known as a heteropolymer. Examples of homopolysaccharides are cellulose, amylose, inulin, chitin, chitosan, amylopectin, glycogen, and pectin. Amylose is a glucose polymer with α(1→4) glycosidic linkages. Amylopectin is a glucose polymer with α(1→4) linkages and branches formed by α(1→6) linkages. Examples of heteropolysaccharides are glucomannan, galactoglucomannan, xyloglucan, 4-O-methylglucuronoxylan, arabinoxylan, and 4-O-Methylglucuronoarabinoxylan.

Polysaccharides can be structurally modified both enzymatically and chemically. Examples of modifications include sulfation, phosphorylation, methylation, O-acetylation, fatty acylation, amino N-acetylation, N-sulfation, branching, and carboxyl lactonization. Glycosaminoglycans are polysaccharides of repeating disaccharides. Within the disaccharides, the sugars tend to be modified, with acidic groups, amino groups, sulfated hydroxyl and amino groups. Glycosaminoglycans tend to be negatively charged, because of the prevalence of acidic groups. Examples of glycosaminoglycans are heparin, chondroitin, and hyaluronic acid.

Polysaccharides that are secreted from cells are known as exopolysaccharides (EPS). Many types of cell walls, in plants, algae, and bacteria, are composed of polysaccharides. The cell walls are formed through excretion of polysaccharides. Organisms excrete polysaccharides that are released from the cells. That is, these molecules are not held in association with the cells as are cell wall polysaccharides. Instead, these molecules are released from the cells. For example, cultures of some species of cyanobacteria excrete exopolysaccharides that are suspended in the culture medium.

### III. Methods of Producing Polysaccharides

### A. Cell Culture Methods: Cyanobacteria

Polysaccharides can be produced by culturing Cyanobacteria. Species of cyanobacteria for use in the disclosure can be identified by morphological and physiological characteristics as well as 16S rDNA analysis.

Cyanobacteria are cultured in a variety of liquid media for polysaccharide production that usually contain, trace elements, a buffer for pH maintenance, and phosphate. Other components can include a fixed nitrogen source such as ammonium or nitrate, and salts such as sodium chloride, particularly for marine cyanobacteria. Examples of trace elements include zinc, boron, cobalt, copper, manganese, and molybdenum usually as salts. Culture condition parameters can be manipulated to optimize total polysaccharide production.

Cyanobacterial species can grow phototrophically in photobioreactors of a variety of types and in open or semi-enclosed ponds using artificial and or natural lighting. Cyanobacteria contain photosynthetic machinery capable of harvesting photons, and transferring energy from photons into fixed chemical energy sources such as monosaccharides. Glucose is a common monosaccharide produced by cyanobacteria by metabolizing light energy and fixing carbon from carbon dioxide. In addition to being a source of chemical energy, monosaccharides such as glucose are also a substrate for the production of polysaccharides.

Cyanobacterial culturing in vessels and photobioreactors is done in small or large liquid volumes. Culturing typically begins with a small number of cells in a small volume (milliliters) and as the cells multiply, they are sequentially transferred into larger and larger volumes. As propagation progresses the increase in biomass is accompanied by an increase in polysaccharide production. For the specific growth methods used see U.S. Pat. No. 9,034,632 B2.

Solid and liquid growth media are generally available from a wide variety of sources, and instructions for the preparation of particular media that is suitable for a wide variety of strains of microorganisms can be found, for example, at the National Center for Marine Algae at Bigelow labs in Maine (see website at ncma.bigelow.org/). Some examples of cyanobacterial culture media include L1, BG-11 (plus sea water) and F/2 (plus seawater). These are general purpose marine media for growing marine cyanobacteria whose composition and method of preparation are found in the following: Guillard, R. R. L. 1975. Culture of phytoplankton for feeding marine invertebrates. pp 26-60. In Smith W. L. and Chanley M. H (Eds.) Culture of Marine Invertebrate Animals. Plenum Press, New York, USA. Guillard, R. R. L. and Hargraves, P. E. 1993. Stichochrysis immobilis is a diatom, not a chrysophyte. Phycologia 32:234-236. Guillard, R. R. L. and Ryther, J. H. 1962. Studies of marine planktonic diatoms. I. Cyclotella nana Hustedt and Detonula confervacea Cleve. Can. J. Microbiol. 8:229-239. See also website at ncma.bigelow.org/algal-recipes.

### B. In Vitro Methods

Polysaccharides can be altered by enzymatic and chemical modification. For example, carbohydrate modifying enzymes can be added to a preparation of polysaccharide and allowed to catalyze reactions that alter the structure of the polysaccharide. Chemical methods can be used to, for example, modify the sulfation pattern of a polysaccharide (see for example Carbohhdr. Polym. 63:75-80 (2000); Pomin V H., Glycobiology. 2005 December; 15 (12): 1376-85; Naggi A., Semin Thromb Hemost. 2001 October; 27 (5): 437-43 Review; Habuchi, O., Glycobiology. 1996 January; 6 (1); 51-7; Chen, J., J. Biol. Chem. In press; Geresh., S et al., J. Biochem. Biophys. Methods 50 (2002) 179-187.).

### C. Polysaccharide Purification Methods

Exopolysaccharides can be purified from cyanobacterial cultures by various methods, including those disclosed herein.

1. Precipitation. For example, polysaccharides can be precipitated by adding compounds such as cetylpyridinium chloride, isopropanol, ethanol, or methanol to an aqueous solution containing a polysaccharide in solution. Pellets or fibers of precipitated polysaccharide can be washed and resuspended in water, buffers such as phosphate buffered saline or Tris, or other aqueous solutions (see, e.g., Farias, W. R. L., et al., J. Biol. Chem. (2000) 275; (38) 29299-29307; U.S. Pat. No. 6,342,367; U.S. Pat. No. 6,969,705).

2. Dialysis. Polysaccharides can also be dialyzed to remove excess salt and other small molecules (see for example Gloaguen, V., et al., Carbohydr Res. 2004 Jan. 2; 339 (1): 97-103; Microbiol Immunol. 2000; 44 (5): 395-400.).

3. Tangential Flow Filtration. Filtration can be used to concentrate polysaccharide and remove salts. For example, tangential flow filtration (TFF), also known as cross-flow filtration, can be used. For an example filtration method see Geresh, Carb. Polym. 50; 183-189 (2002), which discusses use of a MaxCell A/G technologies 0.45 uM hollow fiber filter. Also see for example Millipore Pellicon® devices, used with 100 kD, 300 kD, 1000 kD (catalog number P2C01MC01), 0.1 uM (catalog number P2VVPPV01), 0.22 uM (catalog number P2GVPPV01), and 0.45 uM membranes (catalog number P2HVMPV01). Also see for example NovaSep Kerasep™ ceramic membrane filtration systems with a variety of membrane pore sizes and channel configurations used to differentially separate, for example, biomass from exopolysaccharides or cell lysate components from other disrupted cell materials. It is sometimes desirable that the polysaccharides do not pass through the filter at a significant level. It is also desirable that polysaccharides do not adhere to the filter material. TFF can also be performed using hollow fiber filtration systems using polymeric materials. Non-limiting examples of tangential flow filtration include use of microfiltration with pore sizes of at least about 0.1 micrometer, at least about 0.12 micrometer, at least about 0.14 micrometer, at least about 0.16 micrometer, at least about 0.18 micrometer, at least about 0.2 micrometer, at least about 0.22 micrometer, at least about 0.45 micrometer, or at least about 08 micrometer. In other cases ultrafiltration may be used with pore sizes of at least 15 kD, at least about 50 kD, at least about 150 kD and at least about 300 kD. Examples of suitable pore sizes of tangential flow filtration allow contaminants to pass through but not polysaccharide molecules, or other molecules of interest (e.g. phycocyanin).

4. Ion Exchange Chromatography. Anionic polysaccharides can be purified by anion exchange chromatography. (Jacobsson, I., Biochem J. 1979 Apr. 1; 179 (1): 77-89; Karamanos, N K., Eur J Biochem. 1992 Mar. 1; 204 (2): 553-60).

5. Protease Treatment. Polysaccharides can be treated with proteases to degrade contaminating proteins. In some instances the contaminating proteins are attached, either covalently or noncovalently to polysaccharides. In other instances the polysaccharide molecules are in a preparation that also contains proteins. Proteases can be added to polysaccharide preparations containing proteins to degrade proteins, for example, the protease from Streptomyces griseus can be used (Sigma Aldrich catalog number P5147). After digestion, the polysaccharide is purified from residual proteins, peptide fragments, and amino acids. This purification can be accomplished, for example, by methods listed above such as dialysis, filtration, and precipitation. Heat treatment can also be used to eliminate proteins in polysaccharide preparations (see for example Biotechnol Lett. 2005 January; 27 (1): 13-8; FEMS Immunol Med Microbiol. 2004 Oct. 1; 42 (2): 155-66; Carbohydr Res. 2000 Sep. 8; 328 (2): 199-207; J Biomed Mater Res. 1999; 48 (2): 111-6; Carbohydr Res. 1990 Oct. 15; 207 (1): 101-20). The disclosure thus includes production of an exopolysaccharide comprising separating the exopolysaccharide from contaminants after proteins attached to the exopolysaccharide have been degraded or destroyed. The proteins may be those attached to the exopolysaccharide during culture of a cyanobacterial cell in media, that is first separated from the cells prior to proteolysis or protease treatment. The cells may be those of the genera Synechococcus or Cyanothece as a non-limiting example. In one non-limiting example, a method of producing an exopolysaccharide is provided wherein the method comprises culturing cells of the genera Synechococcus or Cyanothece; separating cells from the culture medium; destroying protein attached to the exopolysaccharide present in the culture medium; and separating the exopolysaccharide from contaminants. In some methods, the contaminant(s) are selected from amino acids, peptides, proteases, protein fragments, and salts.

### 6. Whole Cell Extraction

Because the percentage of polysaccharide as a function of the dry weight of a cell can frequently be in excess of 50%, cell homogenates can be considered partially purified polysaccharide compositions. Purified or isolated polysaccharide compositions contain less than 20% and less than 10% proteins, nucleic acids, intracellular components such as other polysaccharides found intracellularly that are commonly found in microalgae cell homogenate. Cell disruption aids in increasing the amount of solvent-accessible polysaccharide by breaking apart cell walls that are largely composed of polysaccharide.

Intracellular polysaccharides and cell wall polysaccharides can be purified from whole cell mass (see, e.g., U.S. Pat. No. 4,992,540; U.S. Pat. No. 4,810,646; J Sietsma J H., et al., Gen Microbiol. 1981 July; 125 (1): 209-12; Fleet G H, Manners D J., J Gen Microbiol. 1976 May; 94 (1): 180-92).

### D. Drying Methods

After purification by the preceding methods, polysaccharides can be dried using methods such as lyophilization and heat drying (See, e.g., Shastry, S., Brazilian Journal of Microbiology (2005) 36:57-62; Matthews K H., Int J. Pharm. 2005 Jan. 31; 289 (1-2): 51-62. Epub 2004 Dec. 30; Gloaguen, V., et al., Carbohydr Res. 2004 January 2; 339 (1): 97-103). Tray dryers accept moist solid on trays. Hot air (or nitrogen) can be circulated to dry the moist solids. Shelf dryers can also employ reduced (below atmospheric at sea level, such as at about 25 in Hg or less) pressure or vacuum to dry at room temperature when products are temperature sensitive and are similar to a freeze-drier but less costly to use and can be easily scaled-up. In some examples drying in oven tray dryers is performed under vacuum.

### E. Cyanobacterial Cell Disruption (Homogenization) Methods

1. Pressure disruption (French Press). A pressure disrupter pumps a slurry through a restricted orifice valve. High pressure (up to 1500 bar) is applied, followed by an instant expansion through an exiting nozzle. Cell disruption is accomplished by three different mechanisms: impingement on the valve, high liquid shear in the orifice, and sudden pressure drop upon discharge, causing an explosion of the cell. The method is applied mainly for the release of intracellular molecules.

2. Ball Milling (Bead Beating). In a ball mill (or bead beater), cells are agitated in suspension with small abrasive particles. Cells break because of shear forces, grinding between beads, and collisions with beads. The beads disrupt the cells to release biomolecules. The kinetics of biomolecule release by this method is a first-order process.

3. Sonication. Another widely applied method is cell lysis with high frequency sound that is produced electronically and transported through a metallic tip to an appropriately concentrated cellular suspension. The concept of ultrasonic disruption is based on the creation of cavities in cell suspension. Homogenization can also be performed with a Microfluidizer® device (such as the M-110Y Microfluidizer® model, Microfluidics Inc., Newton, Mass.).

4. Blending. Mechanical disruption involves the use of rotating blades. These blades work to grind and disperse cells. Rotor-stator homogenizers are one of the best homogenizing tools used in mechanical disruption, and can homogenize large volumes.

5. Colloid and jet air milling. Cells can also be ground after drying in devices such as a colloid mill or a jet air mill. The jet air mill uses compressed high pressure air to grind material into particles of about 10 um or less.

Homogenization as described herein can increase the amount of solvent-available polysaccharide significantly. For example, homogenization can increase the amount of solvent-available polysaccharide by at least a factor of 0.25, at least a factor of 0.5, at least a factor of 1, at least a factor of 2, at least a factor of 3, at least a factor of 4, at least a factor of 5, at least a factor of 8, at least a factor of 10, at least a factor of 15, at least a factor of 20, at least a factor of 25, and at least a factor of 30 or more compared to the amount of solvent-available polysaccharide in an identical or similar quantity of non-homogenized cells of the same type. One way of determining a quantity of cells sufficient to generate a given quantity of homogenate is to measure the amount of a compound in the homogenate and calculate the gram quantity of cells required to generate this amount of the compound using known data for the amount of the compound per gram mass of cells. The quantity of many such compounds per gram of particular cyanobacterial cells are known. Given a certain quantity of a compound in a composition, the skilled artisan can determine the number of grams of intact cells necessary to generate the observed amount of the compound. The number of grams of cyanobacterial cells present in the composition can then be used to determine if the composition contains at least a certain amount of solvent-available polysaccharide sufficient to indicate whether or not the composition contains homogenized cells, such as for example five times the amount of solvent available polysaccharide present in a similar or identical quantity of unhomogenized cells.

6. Pulsed Electric Field. Pulsed Electric Field (PEF) treatment consists of the application of electric fields of high intensity (>0.1 kV/cm) and short duration (from milliseconds to microseconds) to a product placed between two electrodes. The application of PEF treatments leads to the electroporation or disruption of the cell membranes, and subsequent release of cell contents and cell membrane fragments.

### F. Analysis Methods

Assays for detecting polysaccharides can be used to quantitate starting polysaccharide concentration, measure yield during purification, calculate density of secreted polysaccharide, measure polysaccharide concentration in a finished product, and other purposes. The phenol: sulfuric acid assay detects carbohydrates (see Hellebust, Handbook of Phycological Methods, Cambridge University Press, 1978; and Cuesta G., et al., J Microbiol Methods. 2003 January; 52 (1): 69-73). The 1,6 dimethylmethylene blue assay detects anionic polysaccharides. (see for example Braz J Med Biol Res. 1999 May; 32 (5): 545-50; Clin Chem. 1986 November; 32 (11): 2073-6).

Polysaccharides can also be analyzed through methods such as high pressure liquid chromotograpy (HPLC), size exclusion chromatography, and anion exchange chromatography (see for example Prosky L, Asp N, Schweizer T F, DeVries J W & Furda I (1988) Determination of insoluble, soluble and total dietary fiber in food and food products: Interlaboratory study. Journal of the Association of Official Analytical Chemists 71, 1017±1023; Int J Biol Macromol. 2003 November; 33 (1-3): 9-18).

Polysaccharides can also be detected using gel electrophoresis (see for example Anal Biochem. 2003 Oct. 15; 321 (2): 174-82; Anal Biochem. 2002 Jan. 1; 300 (1): 53-68).

Monosaccharide analysis of polysaccharides can be performed by combined gas chromatography/mass spectrometry (GC/MS) of the per-O-trimethylsilyl (TMS) derivatives of the monosaccharide methyl glycosides produced from the sample by acidic methanolysis (see Merkle and Poppe (1994) Methods Enzymol. 230:1-15; York, et al. (1985) Methods Enzymol. 118:3-40).

The determination of protein concentration may be by use of any known procedure, such as the Lowry assay, the Biuret assay, the Bradford assay, or the bicinchoninic acid (BCA) assay. As a non-limiting example, the BCA assay is based on the formation of a Cu2+-protein complex under alkaline conditions. The Cu2+ is then reduced to Cu1+ where the amount of protein present is proportional to the amount reduction. The reduction has been shown to be mediated by amino acids such as cysteine, cystine, tryptophan, and tyrosine as well as the peptide bond. The result of the assay is a purple-blue complex with Cu1+ under alkaline conditions. The color complex is stable, even in the presence of other components possibly present with the proteins, such as detergents. The amount of reduction can be monitored by absorbance at 562 nm. The BCA assay is sensitive and accurate over a broad range of protein concentrations.

### IV. Compositions

Compositions of the disclosure include a cyanobacterial polysaccharide or homogenate as described herein. In examples relating to polysaccharides, including exopolysaccharides, the composition may comprise a homogenous or a heterogeneous population of polysaccharide molecules, including sulfated polysaccharides as non-limiting examples. Non-limiting examples of homogenous populations include those containing a single type of polysaccharide molecule, such as that with the same structure and molecular weight. Non-limiting examples of heterogeneous populations include those containing more than one type of polysaccharide molecule, such as a mixture of polysaccharides having a molecular weight (MW) within a range of 1000 d to 1000 Kd. In some cases, the polysaccharide has an average molecular weight around 700 Kd. For example, exopolysaccharide from the genus Synechococcus is typically about 100 Kd.

In some examples, a composition of the disclosure may comprise one or more polysaccharides produced by cyanobacteria. In some examples, the cyanobacteria are from the genera Synechococcus or Cyanothece. In some examples, some cyanobacteria grown under different conditions may produce polysaccharides that are different in their monosaccharide composition. Examples include altering the nitrogen source, such as in a non-limiting example, using ammonium instead of nitrate, may change the monosaccharide composition of the polysaccharide that is produced by the cyanobacteria. In some examples, the cyanobacteria are members of the genera Synechococcus or Cyanothece, as a non-limiting example. In some cases, the cell is selected from Synechococcus CCMP 1333, or PCC 7002 or Cyanothece Miami BG 043511.

In some examples, the cyanobacteria have been nutritionally modified resulting in a different profile of monosaccharides in the microalgal polysaccharide.

In some examples, the cyanobacterial polysaccharide comprises 3-9 mole percent arabinose, 0.5-1 mole percent ribose, 10-15 mole percent rhamnose, 7-15 mole percent fucose, 25-35 mole percent xylose, 5-10 mole percent glucuronic acid, 4-12 mole percent mannose, 3-5 mole percent galactose, 16-25 mole percent glucose.

### Cosmeceutical Compositions and Topical Application

### A. General

Compositions, comprising polysaccharides, whole cell extracts, or mixtures of polysaccharides and whole cell extracts, are provided for topical application or non-systemic administration. The polysaccharide may be any one or more of the cyanobacterial polysaccharides disclosed herein, including those produced by a species, or a combination of two or more species. Similarly, a whole cell extract may be that prepared from a cyanobacterial species, or a combination of two or more species. In some examples, polysaccharides, such as exopolysaccharides, and cell extracts from cyanobacteria of the genera Synechococcus and/or Cyanothece are used in the practice of the disclosure.

A composition of the disclosure may comprise from between 0.001% to 100%, 0.01% to 90%, 0.1% to 80%, 1% to 70%, 2% to 60%, 4% to 50%, 6% to 40%, 7% to 30%, 8% to 20%, or 10% polysaccharide, and/or cell extract, by weight.

In an example, a composition of the disclosure may comprise about 0.01%, about 0.02%, about 0.03%, about 0.04%, about 0.05%, about 0.06%, about 0.07%, about 0.08%, about 0.09%, about 0.10%, about 0.11%, about 0.12%, about 0.13%, about 0.14%, about 0.15%, about 0.16%, about 0.17%, about 0.18%, about 0.19% or about 0.20% polysaccharide by weight.

In another example, a composition of the disclosure may comprise from about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1%, about 1.5%, about 2%, about 2.5%, about 3%, about 3.5%, about 4%, about 4.5%, about 5%, about 5.5%, about 6.0%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 65%, about 70%, about 75%, about 80%, about 85%, about 90%, about 95% or about 100% polysaccharide by weight.

In other examples, the composition comprises a carrier suitable for topical administration and/or a preservative suitable for topical administration; of the genera Synechococcus and/or Cyanothece. In further examples, the carrier is suitable for topical administration to humans, such as to human skin or a skin tissue.

In alternative examples, a composition for application to human skin may comprise a polysaccharide isolated from cells from the genera Synechococcus and/or Cyanothece or any other species or strain as disclosed herein. Such a composition may further comprise a carrier and/or preservative suitable for topical administration as described herein. In some cases, the polysaccharide of the composition contains no more than about 10% protein by weight. In other examples, the polysaccharide contains no more than about 5%, no more than about 2%, or no more than about 1% protein by weight. The polysaccharide may also be essentially (e.g., less than 1 wt %), or completely, free of protein, as detectable by assay methods as described herein after treatment to remove protein.

In yet further examples, the polysaccharides are sulfated exopolysaccharides containing at least about 0.05% sulfur, at least about 0.1% sulfur, at least about 0.15% sulfur, at least about 0.2% sulfur, at least about 0.25% sulfur, at least about 0.3% sulfur, at least about 0.35% sulfur, at least about 0.4% sulfur, at least about 0.45% sulfur, at least about 0.5% sulfur, at least about 0.55% sulfur, at least about 0.6% sulfur, at least about 0.65% sulfur, at least about 0.07% sulfur, at least about 0.75% sulfur, at least 0.8% sulfur, at least 0.9% sulfur, at least 1% sulfur, at least 2% sulfur, at least 3% sulfur, at least 4% sulfur, at least 5% sulfur, at least 6% sulfur, at least 7% sulfur, at least 8% sulfur, at least 9% sulfur, or at least 10% sulfur by weight of the polysaccharide. The amount or level of sulfation in the polysaccharides may be analyzed and compared to the amount of sulfates used to culture the cyanobacteria. Thus, the amount or level of sulfation in the polysaccharides of cells grown at less than 50 mM, at about 100 mM, about 200 mM, about 300 mM, about 400 mM, about 500 mM, about 600 mM, or about 700 mM or higher, sulfate (SO₄²⁻) may be determined by routine and repetitive methods disclosed herein. The amount or level of sulfur by weight in the polysaccharides of a sample of cells or cell material may be determined without knowledge of the amount of sulfate used to culture the cells.

In another example, the polysaccharides are subjected to chemical sulfation in order to produce a polysaccharide with a high degree of sulfation. Chemical sulfation processes are well known in the art and are suitable for use in the present disclosure. For example, U.S. Pat. No. 2,599,564 describes methods of chemical sulfation using chlorosulfonic acid and pyridine. U.S. Pat. No. 2,755,275 describes a method of sulfating chitin which uses chlorosulfonic acid or sulfur trioxide as the sulfating agent in an inert solvent such as dichloroethane or other liquid halogenated alkanes. U.S. Pat. No. 4,814,437 describes methods of generating sulfated polysaccharides using pyridine-chlorosulfonic acid, but with a pre-treatment with a reducing agent in order to minimize pyridinium substituents during the preparation of sulfated polysaccharides. This method was shown to achieve from about 13% to about 18% sulfur content in the polysaccharide.

As a further alternative, a composition for topical application to human skin may comprise cyanobacterial cells. The cells may be those from the genera Synechococcus or Cyanothece or any other species or strain as disclosed herein. Optionally, the composition further comprises a carrier and/or preservative suitable for topical administration as described herein. In alternative examples, the cells are homogenized, or otherwise disrupted by common methods (such as by methods described herein) to generate or form a cyanobacterial cell homogenate.

In further examples, the disclosure includes a composition comprising particulate polysaccharides, such as microbeads or nanobeads comprising a disclosed polysaccharide. In some examples the polysaccharide particles are referred to as Heliosugar microspheres. The composition may be for improving the appearance of skin, such as human skin. The polysaccharides may have any level of sulfation described herein. The composition may be sterile and/or non-pyrogenic and optionally substantially free of endotoxins and/or proteins. In other examples, the composition further comprises hyaluronic acid or another agent suitable or desirable for treatment of skin. Non-limiting examples of such an agent include aloe vera, urea, alpha hydroxyl acid, vitamin E, glycyrrhizinic acid, methylsulfonylmethane (MSM), and collagen.

In some examples, the composition comprises a cyanobacterial polysaccharide, wherein the polysaccharide: (a) has been made completely or partially insoluble in water through drying; and (b) has been homogenized or otherwise milled or disrupted to generate particles. The polysaccharide may be of a variety of cyanobacterial cells, such as those of the genera Synechococcus and/or Cyanothece. In some cases, the polysaccharide is contained in a non-aqueous material. As non-limiting examples, the material may be contained in an oil suitable for topical administration, with hexadecanoic acid or oil that is contained in an emulsion as representative examples. The composition may also comprise a carrier and/or preservative suitable for topical administration. The composition may also be substantially free of endotoxins and/or protein as well as sterile and/or non-pyrogenic.

In further examples, the polysaccharide is encapsulated by a timed-release coating, such as one suitable for topical application to human skin. The composition may also comprise a carrier and/or preservative suitable for topical administration. The composition may also comprise a fragrance. The composition may also be substantially free of endotoxins and/or protein as well as sterile and/or non-pyrogenic. In further examples, the polysaccharide is encapsulated by a timed-release coating, such as one suitable for topical application to human skin.

### B. Methods of Cosmetic Enhancement

In a further aspect, the disclosure includes a method to cosmetically enhance skin or its appearance or texture. In some cases, the enhancement is due to increased or improved skin elasticity. The skin may be that of a human being, such as the skin of the face, hands, feet, or other parts of the human body. In other examples, the enhancement may be in the appearance or texture of human lips. The method may comprise administration of a polysaccharide composition suitable for injection into skin or lip tissue to improve the appearance thereof. The composition may be any as described herein suitable for the method of administration or application. In some examples, the injection is made to alleviate or eliminate wrinkles. In other examples, the treatment reduces the visible signs of aging and/or wrinkles. As known to the skilled person, human skin, as it ages, gradually loses skin components that keep skin pliant and youthful looking. The skin components include collagen, elastin, and hyaluronic acid, which have been the subject of interest and use to improve the appearance of aging skin.

The disclosure includes compositions of cyanobacterial polysaccharides, cyanobacterial cell extracts, and cyanobacterial cell homogenates for use in the same manner as collagen and hyaluronic acid. In some examples, the polysaccharides will be those of from a Synechococcus or Cyanothece species. In some examples, the polysaccharides are formulated as a fluid, optionally elastic and/or viscous, suitable for injection. The compositions may be used as injectable dermal fillers as one non-limiting example. The injections may be made into skin to fill-out facial lines and wrinkles. In other examples, the injections may be used for lip enhancement. These applications of polysaccharides are non-limiting examples of non-pharmacological therapeutic methods of the disclosure.

In further examples, the cyanobacterial polysaccharides, cell extracts, and cell homogenates of the disclosure may be co-formulated with collagen and/or hyaluronic acid (such as the Restylane® and Hylaform® products) and injected into facial tissue. Non-limiting examples of such tissue include under the skin in areas of wrinkles and the lips. In an example, the polysaccharide is substantially free of protein. The injections may be repeated as deemed appropriate by the skilled practitioner, such as with a periodicity of about three, about four, about six, about nine, or about twelve months.

The present description is further illustrated by the following examples, which should not be construed as limiting in any way. The contents of all cited references (including literature references, issued patents, published patent applications as cited throughout this application) are hereby expressly incorporated by reference.

### Example 1

### Phototrophic Growth

Synechococcus CCMP 1333, Synechococcus PCC 7002, and Cyanothece Miami BG 043511 were inoculated from stock cultures in L1 medium into autoclaved 1 liter Erlenmeyer flasks containing L1 medium (Guillard, R. R. L. 1975. Culture of phytoplankton for feeding marine invertebrates. pp 26-60. In Smith W. L. and Chanley M. H (Eds.) Culture of Marine Invertebrate Animals. Plenum Press, New York, USA. Guillard, R. R. L. and Hargraves, P. E. 1993. Stichochrysis immobilis is a diatom, not a chrysophyte. Phycologia 32:234-236, and Guillard, R. R. L. and Ryther, J. H. 1962. Studies of marine planktonic diatoms. I. Cyclotella nana Hustedt and Detonula confervacea Cleve. Can. J. Microbiol. 8:229-239. Also see website at ncma.bigelow.org/algal-recipes.

One liter flasks were incubated at room temperature under fluorescent lights in a shaker rotating between 50 and 120 RPM. When the cultures reached stationary phase, cells were transferred to larger volumes of the same medium to propagate the cells into a sufficient volume for commercial harvesting and production of exopolysaccharide (EPS), nominally 1000 L. The final polysaccharide concentration in the culture medium reached between 0.1-5 g/L.

### Example 2

### Purification of Polysaccharide

Cultures of Synechococcus CCMP 1333, Synechococcus PCC 7002 and Cyanothece Miami BG 043511 were grown as described in Example 1. Dense cultures were centrifuged at 15,000×g at 10° C. for 15 minutes to pellet the biomass. Five mL of the supernatant was added to pre-weighed 50 ml conical centrifuge tubes. Forty-five mL of ethanol was added to the polysaccharide-containing supernatant to provide a final concentration of 90% ethanol, and thereby precipitate the EPS. Following precipitation, the solution was centrifuged at 10,000×g for 15 minutes at 10° C. The supernatant was decanted leaving the precipitated EPS pellet. The EPS was dried at 60° C. for a minimum of two hours. The polysaccharide was sufficiently dried when there was no moisture left in the sample as determined by reaching a constant weight of the sample. Alternatively, other samples of the same polysaccharide were isolated from the culture medium using tangential flow filtration (TFF). The largely cell-free fraction after centrifugation was pumped across a membrane of known pore size (TFF), as described above (see section on Tangential Flow Filtration), until the polysaccharide was concentrated on the retentate side of the filter and smaller molecular weight components were passed through the filter. To further remove small molecular weight components, especially salts from the growth medium, deionized water was added to the retentate fraction and pumped across the membrane filter to further isolate the polysaccharide. This diafiltration continued for a minimum of 6 times volume exchange, until the conductivity of the purified polysaccharide fraction was approximately equivalent to physiological saline (e.g. 14.5 mS/cm). Additionally, the viscosity of the permeate and retentate were measured and it was determined that the viscosity of the permeate was equal to physiological saline so it was free of EPS. The purified polysaccharide was then transferred to containers and stored for further processing (e.g., concentrating, drying directly, lyophilization, alcohol precipitation followed by drying, etc.).

### Example 3

### Compositional, Linkage, and Molecular Weight Analysis

Exopolysaccharide (EPS) obtained from a consortium of cyanobacteria including Synechococcus CCMP 1333, Synechococcus PCC 7002 and Cyanothece Miami BG 04351 was evaluated to determine the exopolysaccharide glycosyl composition, glycosyl linkages, molecular weight and total carbohydrate. The cultures were grown as described in Example 1. The biomass was removed by centrifugation, the EPS in the supernatant was purified and concentrated by tangential flow filtration and diafiltration. Various analyses were performed as follows.

### Sample Preparation

250 ml of the purified exopolysaccharide (EPS) was dialyzed against 3 exchanges of water in 6-8 kDa MWCO dialysis tubing (Spectrum Spectra/Por 1). The dialyzed sample was then centrifuged at 4° C. for 25 min at 6000×g to remove any particles, and the supernatant was concentrated to 100 ml using a rotary evaporator. Next, 300 ml of ethanol was added to the concentrated supernatant and this mixture was allowed to stand at room temperature for 2 h. The mixture was then centrifuged again at 4° C. for 25 min at 6000×g. Finally, the precipitate was collected and dialyzed against 3 exchanges of water in 6-8 kDa MWCO dialysis tubing (Spectrum Spectra/Por 1) to remove ethanol. After dialysis the sample was lyophilized.

### Glycosyl Composition Analysis

Glycosyl composition analysis was performed by combined gas chromatography/mass spectrometry (GC/MS) of the per-O-trimethylsilyl (TMS) derivatives of the monosaccharide methyl glycosides produced from the sample by acidic methanolysis as described previously by Santander et al. (2013) Microbiology 159:1471.

Briefly, 1.51 mg of the dried, unprocessed sample was heated with methanolic HCl in a sealed screw-top glass test tube for 18 h at 80° C. After cooling and removal of the solvent under a stream of nitrogen, the samples were treated with a mixture of methanol, pyridine, and acetic anhydride for 30 min. The solvents were evaporated, and the samples were derivatized with Tri-Sil™ HTP Reagent (Thermo Scientific) at 80° C. for 30 min. Following extraction with hexane, GC/MS analysis of TMS methyl glycosides was performed on an Agilent 7890A GC interfaced to a 5975C MSD, using a Supelco Equity-1 fused silica capillary column (30 m×0.25 mm ID).

Composition analysis on the whole exopolysaccharide (EPS) indicates that the sample's carbohydrate component is composed mainly of xylose, rhamnose, mannose, and glucose with other monosaccharides present in significant amounts, see Table 1.

TABLE 1
Compositional analysis results (mol %)

| | mol % |
|---|---|
| Arabinose (Ara) | 2.6 |
| Ribose (Rib) | 0.6 |
| Rhamnose (Rha) | 12.7 |
| Fucose (Fuc) | 8.9 |
| Xylose (Xyl) | 29.9 |
| Glucuronic Acid (GlcA) | 7.7 |
| Mannose (Man) | 9.9 |
| Galactose (Gal) | 5.8 |
| Galacturonic Acid (GalA) | 4.5 |
| Glucose (Glc) | 17.4 |
| SUM | 100.0 |

### Glycosyl Linkage Analysis

1.3 mg of the purified sample was used for linkage analysis. The sample was permethylated by treatment with sodium hydroxide (NaOH) base and iodomethane. First, the sodium hydroxide base was prepared according to the protocol described by Anumula and Taylor (1992) Anal. Biochem. 203:101-108. Briefly, to 200 μL of 50% w/w NaOH was added 400 μL of methanol (MeOH), followed by vortexing. Then 4 mL of dimethyl sulfoxide (DMSO) was added, and the base suspension was vortexed and centrifuged repeatedly up to 5 times to remove residual water and sodium carbonate. After the final extraction, 4 mL of DMSO was added to the NaOH pellet and the suspension was vortexed. Of this final base suspension, 400 μL was added to the sample in DMSO and the mixture was stirred for 2 h. Then, 200 μL of iodomethane was added, and the sample was stirred magnetically at room temperature for 40 min. A second portion of 200 μL iodomethane was added, and the mixture was stirred for an additional 40 min. This is intended to ensure complete methylation. The sample was then extracted with dichloromethane and water. The organic solvent phase and the interphase were separated and dried under N₂ stream.

The sample was dialyzed against 0.05% NaN₃ using 100-kDa dialysis tubing. The retentate was lyophilized, and 0.84 mg of the purified sample was dissolved in 400 μL DMSO for linkage analysis. The sample was permethylated by treatment with sodium hydroxide (NaOH) base and iodomethane. First, the sodium hydroxide base was prepared according to the protocol described by Anumula and Taylor (1992) Anal. Biochem. 203:101-108. Briefly, to 200 μL of 50% w/w NaOH was added 400 μL of methanol (MeOH), followed by vortexing. Then 4 mL of dimethyl sulfoxide (DMSO) was added, and the base suspension was vortexed and centrifuged repeatedly up to 5 times to remove residual water and sodium carbonate. After the final extraction, 4 mL of DMSO was added to the NaOH pellet and the suspension was vortexed. Of this final base suspension, 400 μL was added to the sample in DMSO and the mixture was stirred for 2 h. Then, 200 μL of iodomethane was added, and the sample was stirred magnetically at room temperature for 40 min. A second portion of 200 μL iodomethane was added, and the mixture was stirred for an additional 40 min. This is intended to ensure complete methylation. The sample was then extracted with dichloromethane and water. The organic solvent phase and the interphase were separated and dried under N₂ stream.

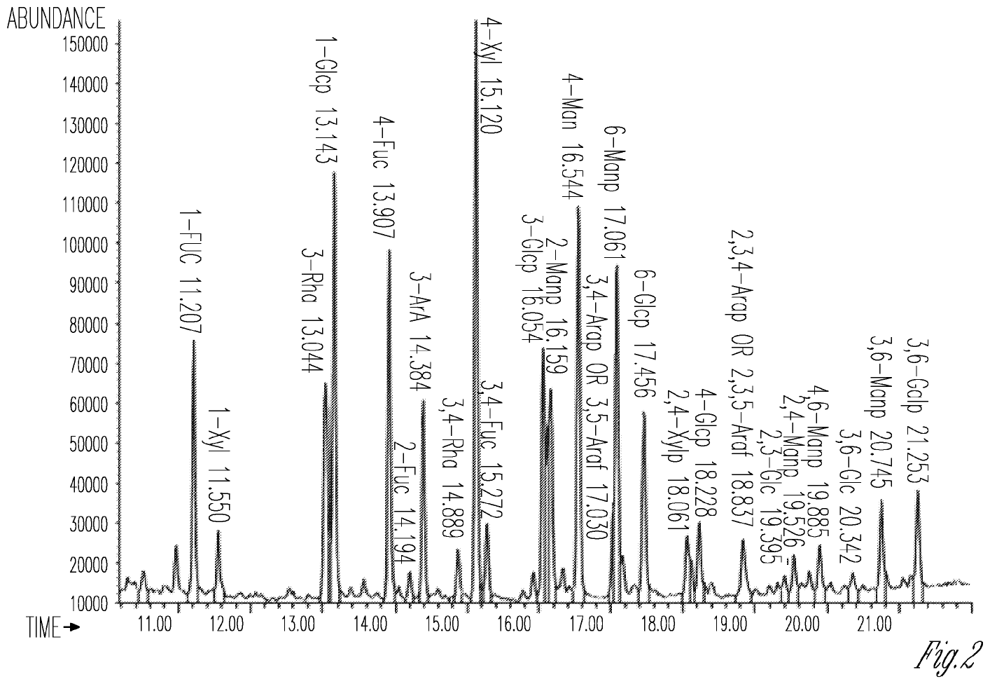

The relative percentage of each glycosyl linkage identified are listed in Table 2 and the glycosyl linkage chromatogram is shown in FIG. 2. The cyanobacterial EPS sample consists of many types of glycosyl linkages with relatively evenly distributed types. Among these linkages, 4-Xylp (11.45%), t-Xylp (5.12%), 4-Manp (8.98%), 6-Manp (7.38%), 2-Manp (4.94%), 4-Fucp (8.46%), 2-Fuc (7.09%), t-Glcp (6.78%), 3-Glcp (5.94%), and 6-Gclp (4.64%) are the major glycosyl types.

TABLE 2
Relative percentage of each detected linkage.

| | Relative |
|---|---|
| | Area |
| | Percentage |
| Glycosyl Linkage Residue | (%) |
| Terminal Fucopyranosyl Residue (t-Fucp) | 1.08 |
| Terminal Arabinofuranosyl Residue (t-Araf) | — |
| Terminal Xylopyranosyl Residue (t-Xylp) | 5.12 |
| 3-Linked Rhamnopyranosyl Residue (3-Rhap) | 1.74 |
| Terminal Glucopyranosyl Residue (t-Glcp) | 6.78 |
| 4-Linked Fucopyranosyl Residue (4-Fucp) | 8.46 |
| 2-Linked Fucopyranosyl Residue (2-Fucp) | 7.09 |
| 3-Linked Arabinofuranosyl Residue (3-Araf) | 0.73 |
| Terminal Glucofuranosyl Residue (t-Glcf) | 4.10 |
| 3,4-Linked Rhamnopyranosyl Residue (3,4-Rhap) | 1.21 |
| 4-Linked Xylopyranosyl Residue (4-Xylp) | 11.45 |
| 3,4-Linked Fucopyranosyl Residue (3,4-Fucp) | 1.85 |
| 3-Linked Glucopyranosyl Residue (3-Glcp) | 5.94 |
| 2 Linked Mannopyranosyl Residue (2-Manp) | 4.94 |
| 4-Linked Mannopyranosyl Residue (4-Manp) | 8.98 |
| 3,4-Linked Arabinopyranosyl or | 1.29 |
| 3,5-Linked Arabinofuranosyl Residue | |
| (3,4-Arap or 3,5-Araf) | |
| 6-Linked Mannopyranosyl Residue (6-Manp) | 7.38 |
| 6-Linked Glucopyranosyl Residue (6-Glcp) | 4.64 |
| 2,4-Linked Xylopyranosyl Residue (2,4-Xylp) | 1.84 |
| 4-Linked Glucopyranosyl Residue (4-Glcp) | 2.14 |
| 2,3,4-Linked Arabinopyranosyl or | 2.34 |
| 2,3,5-Linked Arabinofuranosyl Residue | |
| (2,3,4-Arap or 2,3,5-Araf) | |
| 2,3-Linked Glucopyranosyl Residue (2,3-Glcp) | 0.74 |
| 2,4-Linked Mannopyranosyl Residue (2,4-Manp) | 1.47 |
| 4,6-Linked Mannopyranosyl Residue (4,6-Manp) | 1.79 |
| 3,6-Linked Glucopyranosyl Residue (3,6-Glcp) | 1.14 |
| 2,6-Linked Glucopyranosyl and | — |
| 4,6-Linked Glucopyranosyl Residue | |
| (2,6-Glcp and 4,6-Glcp) | |
| 3,6-Linked Mannopyranosyl Residue (3,6-Manp) | 2.77 |
| 3,6-Linked Galactopyranosyl Residue (3,6-Galp) | 3.11 |
| Total | 100.12 |

Note that the total Area % shown in Table 2 may not add to exactly 100% due to rounding.

### Size Exclusion Chromatography (SEC) Analysis

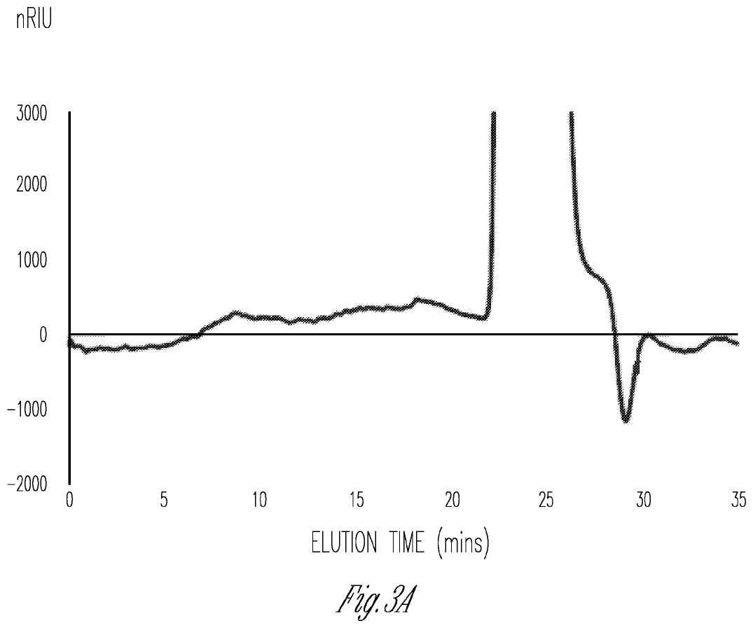

The method used for SEC of the analyte was adapted from Yanagisawa, Shibata and Isogai (2005), Cellulose, 12, 151-158. The SEC system used was Agilent 1260 Infinity, which consists of a quaternary pump (G1316A) with a built-in degasser, thermostatically controlled column compartment (G1314A), high performance autosampler (G1329B), a fraction collector (G1364C), a variable wavelength detector (G1314F) and a refractive index detector (G1362A). The SEC chromatogram of the analyte and the dextran standards (10, 66.9, 167, 511, 759.4 and 1185 kDa) are shown in FIG. 3. The largest dextran standard, 1185 kDa, had a peak maximum at 16.135 min. Due to solubility issues with the sample and presence of non-carbohydrate materials a precise measure of molecular weight was not obtained. However, the data suggest a molecular weight >700 kDa.

FIG. 3: Size exclusion chromatogram of (A) Sample M and (A) six dextran standards; 10, 66.9, 167, 511, 759.4 and 1185 kDa.

### Phenol/Sulfuric Acid Assay

A portion of the dried sample was dissolved in Milli-Q H₂O to a concentration of 0.1 mg/ml. Glucose standards were prepared with concentrations ranging from 20 μg/ml to 200 μg/ml. One hundred microliters of each of these standards, along with the sample and a blank of Milli-Q H₂O was transferred to borosilicate glass culture tubes. To this was added 100 μl of 5% phenol and 500 μl of concentrated sulfuric acid in each tube. The tubes were then heated in a 100° C. water bath for 10 min and then allowed to cool before being transferred to a 96-well plate. The absorbance was measured at 490 nm.

The material remaining in the centrifugal filter after filtration was resuspended in 100 μ—L Milli-Q H₂O. Aliquots of 10 μL each of this material and the filtrate were transferred to a 96-well plate, along with glucose standards ranging in concentration from 0.05 mg/mL to 1 mg/mL. Concentrated sulfuric acid (200 μL) and 40 μl 5% phenol were added to each well, and the plate was heated on a 100° C. heating block for 5 min. The plate was allowed to cool before measuring the absorbance at 490 nm.

The phenol/sulfuric acid assay indicated a total carbohydrate content as glucose equivalents by weight is 32%.

### Toxicity and Efficacy Testing of Extracellular Polysaccharides

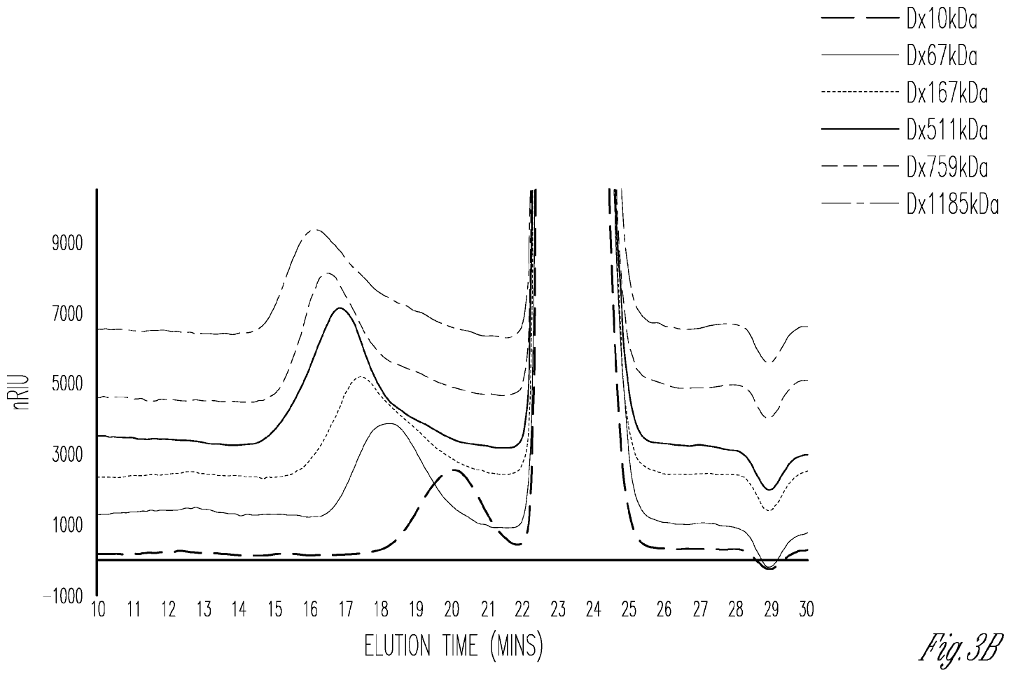

Testing of the extracellular polysaccharides from the consortium for toxicity using the MTT (3-(4,5-dimethylthiazol-2-yl)-2,5-diphenyltetrazolium bromide, a tetrazole) assay for cell metabolic activity illustrated that the extracellular polysaccharides are not toxic (FIG. 4).

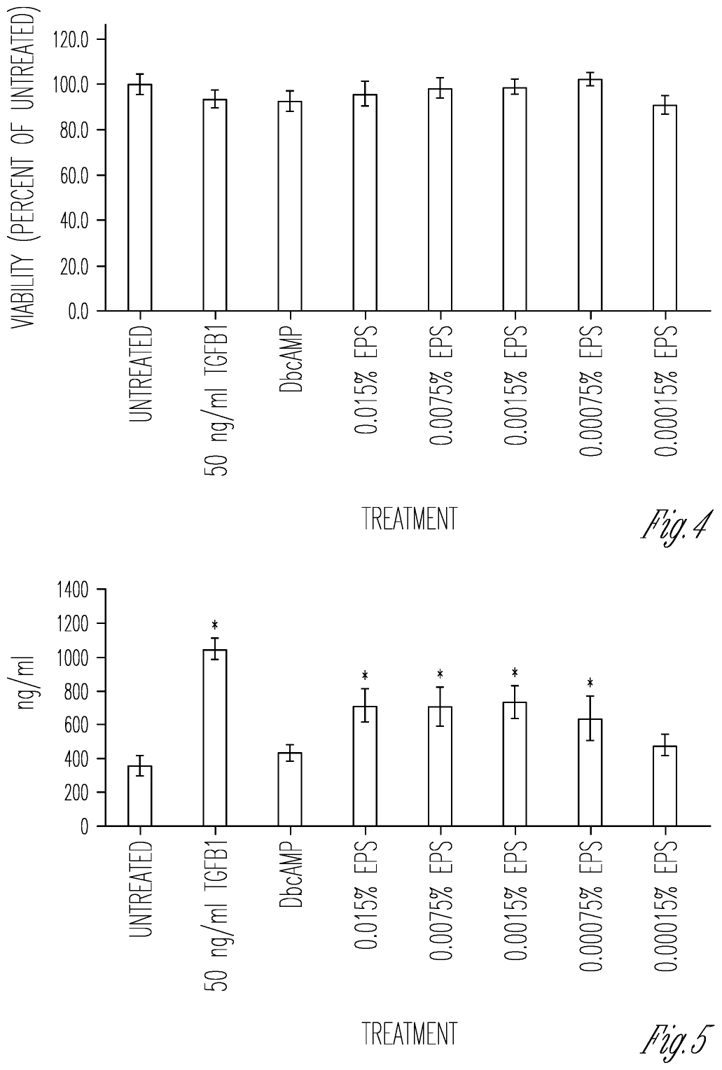

Cultured human dermal fibroblasts were used to assess the ability of the EPS to affect type I collagen synthesis. The EPS significantly stimulated the production of type I collagen at 0.015%, 0.0075%, 0.0015% and 0.00075% wt/vol. (FIG. 5).

### Exemplary Aspects

The following exemplary embodiments are provided, the ordering of which is not to be construed as designating levels of importance:

Aspect 1 provides a composition comprising one or more isolated exopolysaccharide from cyanobacteria of the genus Synechococcus and/or Cyanothece, wherein the monosaccharide content of the exopolysaccharide comprises 3-9 mole percent arabinose, 0.5-1 mole percent ribose, 10-15 mole percent rhamnose, 7-15 mole percent fucose, 25-35 mole percent xylose, 5-10 mole percent glucuronic acid, 4-12 mole percent mannose, 3-5 mole percent galactose, 16-25 mole percent glucose.

Aspect 2 provides the composition of Aspect 1, wherein the exopolysaccharide is milled to provide exopolysaccharide particles having an average particle size of in a range of from about 400 microns and about 0.1 microns.

Aspect 3 provides the composition of Aspect 2, wherein the average particle size of the exopolysaccharide particles is in a range of from about 50 microns and about 200 microns.

Aspect 4 provides the composition of Aspect 2, wherein the exopolysaccharide particles are partially soluble in water.

Aspect 5 provides the composition of Aspect 1, wherein the exopolysaccharide is suspended in water to have a viscosity of in a range of from about 1 cP and about 1000 cP.

Aspect 6 provides the composition of Aspect 1, wherein the exopolysaccharide(s) are from Synechococcus CCMP 1333, or Synechococcus PCC 7002, or Cyanothece Miami BG 043511.

Aspect 7 provides the composition of Aspect 2, wherein the exopolysaccharide particles increase in volume upon contact with water compared to the anhydrous volume of the exopolysaccharide particles.

Aspect 8 provides the composition of Aspect 7, wherein the exopolysaccharide particles at least double in volume.

Aspect 9 provides the composition of Aspect 1, 2, or 5 wherein the exopolysaccharide(s) are formulated with at least one excipient suitable for topical administration.

Aspect 10 provides a method of improving the appearance of skin comprising applying the composition of any one of Aspects 1-9 to human skin.

Aspect 11 provides a method of preparing exopolysaccharide, comprising:

a. culturing a cyanobacteria of the genera separately or combined Synechococcus CCMP 1333, Synechococcus PCC 7002, and Cyanothece Miami BG 043511 phototrophically in a culture medium;

b. isolating exopolysaccharide from the culture medium;

c. concentrating the isolated exopolysaccharide(s); and

d. formulating the expolysaccharide as either annealed particles or in aqueous solution with at least one excipient suitable for topical administration.

Aspect 12 provides the method of Aspect 11, wherein said exopolysaccharide is isolated from the culture medium by alcohol precipitation.

Aspect 13 provides the product made by the process of Aspect 11.

Aspect 14 provides a method of preparing a composition for topical application, the method comprising:

a. culturing a cyanobacteria of the genera Synechococcus CCMP 1333, Synechococcus PCC 7002, and Cyanothece Miami BG 043511, separately or combined phototrophically in a culture medium;

b. isolating exopolysaccharide from the culture medium;

c. drying the isolated exopolysaccharide;

d. milling the dried exopolysaccharide to a size of between about 400 microns and about 0.1 microns to prepare exopolysaccharide particles;

e provides annealing the exopolysaccharide particles; and

f. formulating the annealed expolysaccharide particles with at least one excipient suitable for topical administration.

Aspect 15 provides a method of preparing exopolysaccharide particles, the method comprising:

a. culturing a microalgae of the genera Synechococcus CCMP 1333, Synechococcus PCC 7002, and Cyanothece Miami BG 043511, separately or combined phototrophically in a culture medium;

b. isolating exopolysaccharide from the culture medium;

c. processing the isolated exopolysaccharide by heating; chemical crosslinking; chemical dehydration through displacement of bound water by an alcohol; precipitation from solution using (i) an alcohol, (ii) a ketone, or (iii) pH (from 7 to >11); or coating of particles by microencapsulation; and milling the dried exopolysaccharide to a size of between about 400 microns and about 0.1 microns to prepare exopolysaccharide particles.

Aspect 16 provides a cosmetic skin composition comprising a homogeneous mixture of water, a topical cosmetic emollient in an amount ranging from 0.5 to about 99 weight percent of the water weight and a water solubilized/dispersible polysaccharide in, an amount in a range of from about 0.000015 to about 10 weight percent of the water weight, said polysaccharide having a molecular weight in a range of from about 10,000 to about 8×10⁶ daltons or from about 19,000 to about 7×10⁶ daltons.

Aspect 17 provides the cosmetic skin composition according to Aspect 16 wherein the polysaccharide has an average molecular weight from about 10,000 to about 8×10⁶ daltons or from about 19,000 to about 7×10⁶ daltons.

Aspect 18 provides the cosmetic composition according, to Aspect 17 wherein the amount of polysaccharide ranges from about 0.00001 to about 3 weight percent, said weight percent being based upon the total amount of water contained in the cosmetic composition.

1. A composition comprising one or more isolated exopolysaccharide from cyanobacteria of the genus Synechococcus and/or Cyanothece, wherein the monosaccharide content of the exopolysaccharide comprises 3-9 mole percent arabinose, 0.5-1 mole percent ribose, 10-15 mole percent rhamnose, 7-15 mole percent fucose, 25-35 mole percent xylose, 5-10 mole percent glucuronic acid, 4-12 mole percent mannose, 3-5 mole percent galactose, 16-25 mole percent glucose.

2. The composition of claim 1, wherein the exopolysaccharide is milled to provide exopolysaccharide particles having an average particle size of in a range of from about 400 microns and about 0.1 microns.

3. The composition of claim 2, wherein the average particle size of the exopolysaccharide particles is in a range of from about 50 microns and about 200 microns.

4. The composition of claim 2, wherein the exopolysaccharide particles are partially soluble in water.

5. The composition of claim 1, wherein the exopolysaccharide is suspended in water to have a viscosity of in a range of from about 1 cP and about 1000 cP.

6. The composition of claim 1, wherein the exopolysaccharide(s) are from Synechococcus CCMP 1333, or Synechococcus PCC 7002, or Cyanothece Miami BG 043511.

7. The composition of claim 2, wherein the exopolysaccharide particles increase in volume upon contact with water compared to the anhydrous volume of the exopolysaccharide particles.

8. The composition of claim 7, wherein the exopolysaccharide particles at least double in volume.

9. The composition of claim 1, wherein the exopolysaccharide(s) are formulated with at least one excipient suitable for topical administration.

10. A method of improving the appearance of skin comprising applying the composition of claim 1 to human skin.

11. A method of preparing exopolysaccharide, comprising:
a. culturing a cyanobacteria of the genera separately or combined Synechococcus CCMP 1333, Synechococcus PCC 7002, and Cyanothece Miami BG 043511 phototrophically in a culture medium;
b. isolating exopolysaccharide from the culture medium;
c. concentrating the isolated exopolysaccharide(s); and
d. formulating the expolysaccharide as either annealed particles or in aqueous solution with at least one excipient suitable for topical administration.

12. The method of claim 11, wherein said exopolysaccharide is isolated from the culture medium by alcohol precipitation.

13. The product made by the process of claim 11.

14. A method of preparing a composition for topical application, the method comprising:
a. culturing a cyanobacteria of the genera Synechococcus CCMP 1333, Synechococcus PCC 7002, and Cyanothece Miami BG 043511, separately or combined phototrophically in a culture medium;
b. isolating exopolysaccharide from the culture medium;
c. drying the isolated exopolysaccharide;
d. milling the dried exopolysaccharide to a size of between about 400 microns and about 0.1 microns to prepare exopolysaccharide particles;
e. annealing the exopolysaccharide particles; and
f. formulating the annealed expolysaccharide particles with at least one excipient suitable for topical administration.

15. A method of preparing exopolysaccharide particles, the method comprising:
a. culturing a microalgae of the genera Synechococcus CCMP 1333, Synechococcus PCC 7002, and Cyanothece Miami BG 043511, separately or combined phototrophically in a culture medium;
b. isolating exopolysaccharide from the culture medium;
c. processing the isolated exopolysaccharide by heating; chemical crosslinking; chemical dehydration through displacement of bound water by an alcohol; precipitation from solution using (i) an alcohol, (ii) a ketone, or (iii) pH (from 7 to >11); or coating of particles by microencapsulation; and milling the dried exopolysaccharide to a size of between about 400 microns and about 0.1 microns to prepare exopolysaccharide particles.

16. A cosmetic skin composition comprising a homogeneous mixture of water, a topical cosmetic emollient in an amount ranging from 0.5 to about 99 weight percent of the water weight and a water solubilized/dispersible polysaccharide in, an amount in a range of from about 0.000015 to about 10 weight percent of the water weight, said polysaccharide having a molecular weight in a range of from about 10,000 to about 8×10⁶ daltons or from about 19,000 to about 7×10⁶ daltons

17. The cosmetic skin composition according to claim 16 wherein the polysaccharide has an average molecular weight from about 10,000 to about 8×10⁶ daltons or from about 19,000 to about 7×10⁶ daltons.

18. The cosmetic composition according, to claim 17 wherein the amount of polysaccharide ranges from about 0.00001 to about 3 weight percent, said weight percent being based upon the total amount of water contained in the cosmetic composition.
